(19) United States
(12) Patent Application Publication
(10) Pub. No.: US 2011/0295159 A1
(43) Pub. Date: Dec. 1, 2011

(54) Method and Apparatus for an Implantable Inertial-Based Sensing System for Real-Time, In Vivo Detection of Spinal Pseudarthrosis and Adjacent Segment Motion

(75) Inventors: Yehoshua Shachar, Santa Monica, CA (US); Thomas Chen, La Canada, CA (US); Winston Wu, Alhambra, CA (US); Brett Jordan, Los Angeles, CA (US); Herwin Chan, Los Angeles, CA (US); Paladin Luboff, Santa Monica, CA (US); Kyle Zimmerman, Los Angeles, CA (US)

(73) Assignee: PHARMACO-KINESIS CORPORATION, Inglewood, CA (US)

(21) Appl. No.: 12/787,286

(22) Filed: May 25, 2010

(51) Int. Cl. A61B 5/103; A61F 2/44

(52) U.S. Cl. 600/594; 623/17.11

(57) ABSTRACT

A vertebral processor designed to collect and interpret data from multiple surgically implanted accelerometers. Each accelerometer is surgically implanted into a vertebra of a patient utilizing a bone screw. Additional accelerometers are implanted in adjacent vertebrae. The data from the accelerometers is compared by an algorithm to determine the relative movement of the accelerometers implanted in adjacent vertebrae. Data is generated via the algorithm and compared against the expected behavior of the surgically implanted accelerometers as if they were connected to a rigid body, thus determining the level of success of a spinal fusion procedure for those adjacent segments. The apparatus may be utilized with or without spinal stabilization hardware, and with or without fusion cages or artificial discs. The vertebral processor is supplemented by an external system worn by the patient, which provides for an inductive charging power source and for data transfer.

### BACKGROUND OF THE INVENTION

1. Field of the Invention

The invention relates to the field of implantable biosensor systems, specifically to remotely powered and controlled accelerometer devices that are surgically implanted into adjacent vertebrae for the purpose of determining the success of spinal fusion procedures.

2. Description of the Art

Back pain is one of the most commonly treated medical conditions in the United States today and is second only to the common cold as the most common reason for physician visits. Back pain not only has a profound effect on patients, it has an exceedingly high societal cost. Back pain is the second most common cause of lost productive time for pain disorders and results in the largest amount of total lost time. It has been stated that of all medical conditions, back pain results in the most lost productivity. It is estimated that the direct and indirect costs associated with the disorder are approximately 50 billion dollars per year in the United States alone. Although back pain is multifactorial, degenerative disc disease is often involved in the pathogenesis and subsequent propagation of lower back pain.

The disc space is composed of a disc and surrounding annulus fibrosis. As the disc degenerates, hypertrophy is increased in order to support the body weight, eventually leading to back pain. The achievement of a bony fusion is necessary for bony regeneration and healing, and is crucial to the success of many orthopedic procedures. It is estimated that more than 300,000 spinal fusion procedures are performed each year in the United States alone.

Spinal surgery is currently composed of two main facets: decompression and reconstruction. Decompression removes ligaments, bone, or tumor tissues that may be causing pressure on the spinal cord or nerve root. Reconstruction consists of deformity correction and fusion of vertebrae. Fusion is performed with a variety of instrumentations that are used to hold the spine in a certain position. The actual process of bony fusion, however, is biological and consists of osteoblasts crossing from one bone to another, forming a bridge of bone called the fusion mass. Spinal fusion can be performed via an anterior approach, involving procedures such as interbody fusions and corpectomies. Posterior fusions generally involve pedicle screws and connecting rods to hold the fusion in position. In both cases, the instrumentation is there to accelerate and enable a bony biological fusion to take place.

Traditionally, spinal fusions have been performed by decorticating the host bone, and laying down autograft from the iliac crest to achieve a bony, in situ, fusion. With the advent and popularity of instrumentation for spinal fusions (e.g. pedicle screws), adjacent segments could now be held together, immobilizing them, thus enhancing the fusion rate. However, even with the use of iliac crest autograft and spinal fixation, the development of a biologic fusion (i.e., new bone formation at the fusion site) is not consistent. Spinal fixation allows for short term mechanical stabilization, but lacks the capacity to produce a consistent biologic fusion. Further, determining the short-and-long term success of spinal fixation procedures is hampered by sub-optimal testing procedures which generally involve infrequent x-ray techniques.

After the surgery is performed, the patient typically wears an external brace for three months. This external brace is worn to remind the patient not to bend or twist too much, and also to give the patient extra support. Regular x-rays are typically taken one month and three months after surgery. If a fusion mass is demonstrated after three months, the patient is allowed to take the brace off, and to start physical therapy. The physical therapy is performed to strengthen the back and redevelop paraspinous muscles, which have been weakened by the surgery and by the inactivity of the immobilization in the brace after surgery.

If the patient develops pain several months after a fusion procedure, there are a variety of possibilities that must be investigated. The differential diagnosis includes: 1) residual compression, 2) pseudarthrosis, 3) adjacent segment effects, and 4) chronic/subacute infection. Residual compression can usually be ruled out with anatomical studies such as a MRI scan or CT-myelogram. Subacute/chronic infections are rare, but may be suggested by increased temperature, high White Blood Cell (WBC) count, or high erythrocyte sedimentation rate (ESR). Pseudarthrosis is determined by plain x-rays or CT-scans demonstrating lack of a bony fusion. Adjacent segment effects are presumed to have occurred from the fused segment of spine acting as a lever arm on the next free segment, causing increased stress and pain. In both cases, motion is assumed on the basis of static films such as x-rays, CT-scans, or MRI scans.

Current techniques for detection of intervertebral fusion success are limited to x-rays, CT-scans, MRI scans, and, more recently, mechanical strain sensors attached to the rod that joins two pedicle screws. X-ray and CT-scan detection methods for failed fusion procedures are not optimal because they provide infrequent and controlled data. The patient must visit the hospital to perform these procedures, and the imaging is of one moment in time. Mechanical strain sensors can provide continual feedback. However, they are located on the connection rod between stabilization screws (e.g. pedicle screws), and not on the vertebrate where sensory data is needed. Data regarding the mechanical strain placed on aspects of the hardware implants utilized to stabilize a spinal fusion treatment do not directly inform physicians as to the relative movement of the patient's vertebra. This can lead to inaccurate data from the strain sensor, for example, if the pedicle screw were to fracture.

### BRIEF SUMMARY OF THE INVENTION

The following summary of the invention is provided to facilitate an understanding of some of the innovative features unique to the present invention and is not intended to be a full description. A full appreciation of the various aspects of the invention can be gained by taking the entire specification, claims, drawings, and abstract as a whole. Additional objects and advantages of the current invention will become apparent to one of ordinary skill in the art upon reading the specification.

The illustrated embodiments of the invention described herein is directed specifically at spine fusion treatments. Different devices will be tailor made for various instrumentalities depending on location of placement (e.g., posterior versus anterior spinal column, or intervertebral disc). Moreover, the placement of the vertebral body motion sensors on the vertebrae will be different, depending on the disease process to be treated. For instance, in a degenerating disc, the sensors may be used along with the spine stabilization hardware to detect the loosening of the pedicle screws. In the case where the interbody disc is removed during surgery, it may be placed along with an interbody fusion pump. It may also be placed separately on the anterior or posterior spinal column.

To detect spinal pseudarthrosis, tracking of the change in orientation is not required. Only the accelerometers are required to determine whether the two spine segments are still fused together. Our approach covers at least two situations. For the situation where the patient is moving rapidly, such as brisk walking, we use vibration monitoring to detect change in relative acceleration between two fused vertebrae. If the vertebrae are fused, they form a rigid body and the relative acceleration between the sensors is zero. For the situation where patient is moving slowly, such as bending, we use tilt monitoring to detect relative angular change respect to the gravity. If the vertebrae are fused, the relative tilt angle between the two sensors is zero.

The implanted accelerometer allows information about the instrumentation to be passed on to the treating surgeon and the physical therapist. The goal is to create instrumentation that can be implanted in the bone adjacent to the instrumentation. The accelerometer will notify the physician that there is motion within the fusion. If the bony fusion does not occur, then the implanted instrumentation will weaken, the screws will become loose, and the patient develops a pseudarthrosis. When a complete solid fusion occurs, however, the instrumentation will remain in place. An accelerometer at the segments above and below will let the physician know over time if there is increased motion that may lead to stress and pressure on the adjacent facet segments, accounting for pain.

The accelerometer is implanted so that it forms a geometric configuration that can be used to determine the position of the patient. This will allow the physical therapist the ability to provide the patient with the best bending motion to strengthen the back without stressing the rest of the fusion. In essence, with this implanted accelerometer, the physical therapist and the physician are now the bio-mechanist of the human skeletal system.

The illustrated embodiments of the disclosed invention is capable of transmitting information about the fusion device and the spinal segments adjacent to the instrumentation. This device is capable of providing instantaneous feedback about the fusion and converting “dumb” titanium fixation into an intelligent sensor device.

The present disclosure also describes an apparatus comprising architecture for remote monitoring of the vertebral movement. Furthermore, the illustrated device provides the physician with a means to ascertain the success of the fusion and instrumentation without the need for repeated imaging as characterized by differential motion. The fused bone segments are intended to act as one rigid body and the difference in acceleration parameters should be negligible. The illustrated embodiment of the invention employs a mathematical model that enables the detection in vivo using external inductive power and a communication belt as well as a mathematical foundation of the process by extending and generalizing the model to most movement forms encountered by the patient. The detection algorithm runs on an implanted microcontroller and the thresholds can be modified wirelessly by the communication belt. The model is represented as a set of equations and is composed of two conditions. For a repetitive and quick movement such as brisk walking, the model uses vibration monitoring to discern whether the two vertebral segments are one rigid body by detecting the difference in acceleration between the two segments. For a slow motion such as bending or the lifting of an object, the model uses tilt angle monitoring to differentiate the relative tilt angles between the bone segments with respect to gravity. The results obtained are assumed to be qualitatively similar to the actual physiological ones and quantitatively close to some available experimental data noted by clinical studies.

An object of the illustrated embodiments of the invention is to provide the patient and physician with a feedback alert after detection of a loose or broken pedicle screw. Further actions, such as radiographic imaging or surgery, can be planned or performed based on the degree of the looseness as detected by the sensors. The device may be used in conjunction with interbody posterolateral fusion pump systems or installed separately for anterior fusion monitoring.

In another embodiment, the illustrated invention comprises inductive coils that can be energized from an inductive power source located on an external device worn as a belt. The detector and its electronics will remain passive until power is received from the control units via inductive coils. The reader unit communicates to the implant over the inductive link for the on/off control of the detector. This link can be utilized to modify the detection thresholds. When energized from the inductive coupling, the detector provides power to a plurality of motion sensors. The reader processes the sensor values and transmits the results via the said inductive communication link.

In one embodiment, two separate inductive coils are utilized in the invention in both the implant and the external device worn as a belt. This configuration, allows for one coil would provide transmission/reception of inductive power, and the second to provide transmission/reception of data.

In one embodiment of the illustrated invention, four motion sensors are placed in the L2-L5 lumbar vertebrae, one within the detector unit on L3 or L4, and the other three on each of the adjacent vertebra. The motion sensors provide information on relative tilt motion between the two vertebrae above and below the fusion joint. The relative tilt motion between these two spinal bone segments, which is sent via the inductive communication link, helps a physician monitor the increased range of motion that might lead to adjacent segment disease. When the two bone segments are totally fused, the range of motion in terms of tilt angle between the adjacent segment and the fused vertebrae increase. The risk of adjacent segment disease could be minimized by providing the patient with a feedback alert when the tilt angle exceeding threshold level is detected.

It is yet another object of the illustrated embodiments of the invention is to facilitate restoration of health to the patient and assist in physical rehabilitation. The system actively detects the tilt angles and provides warning when the measurement exceeds the threshold. This new sensing/feedback method provides peace of mind when exercising for physical rehabilitation and could lead to a healthier patient.

In summary, the illustrated embodiments of the invention include an apparatus for sensing relative vertebral movement within a spine of a patient, which includes an implantable electronics assembly coupled to at least one of a plurality of vertebrae within the spine for monitoring spatial orientation of at least one of the plurality of vertebrae, and an external system disposable proximate to the implantable electronics assembly and comprising an induction link and a circuit communicated to the implantable electronics assembly through the induction link for communicating data related to the spatial orientation of at least one of the plurality of vertebrae.

The implantable electronics assembly includes a vertebral processor, and at least two sensors or accelerometers coupled to the vertebral processor and to two corresponding vertebrae within the spine.

The vertebral processor is coupled to a vertebra within the spine of the patient.

The vertebral processor is coupled to a stabilization rod coupled to at least two adjacent vertebrae within the spine of the patient.

The implantable electronics assembly includes an implantable data circuit and an implantable induction coil coupled to the implantable data circuit. The external system includes a data receiver circuit, and an external induction coil coupled to the data receiver circuit. The implantable data circuit transmits data received by the implantable electronics assembly to the data receiver circuit through electromagnetic coupling of the external and implantable induction coils. The data receiver circuit transmits instructions to the implantable data circuit and implantable electronics assembly through electromagnetic coupling of the external and implantable induction coils.

The implantable electronics assembly includes a power regulator circuit, and at least one power induction coil coupled to the power regulator. The external system includes a power generation circuit, and at least one power induction coil coupled to the power generation circuit. The power generation circuit transmits power through the power induction coil to the power regulator circuit. The power regulator circuit receives power from the power generator circuit through the power induction coil to power the implantable electronics assembly.

The external system includes a strap, and a reader unit coupled to the strap which is worn by a patient so that the reader unit is proximate to the implantable electronics assembly implanted within the patient.

The reader unit includes a power generation circuit and a data receiver circuit coupled to the external induction coil.

The two sensors or accelerometers are coupled to at least one vertebra above and at least one vertebra below the interbody fusion cage or artificial disk.

The scope of the illustrated embodiments of the invention also includes a method for monitoring relative movement of vertebrae in a spine of a patient including the steps of providing an implantable electronics assembly for coupling to a plurality of vertebrae within the spine capable of sensing movement of the plurality of vertebrae relative to one another, providing an external system for proximate monitoring of the patient, collecting data of the relative movement of the vertebrae sensed by the implantable electronics assembly, and calculating the relative orientation of the vertebrae from the collected data.

The method further includes the steps of coupling a vertebral processor directly or indirectly to a vertebra within the spine, coupling a plurality of accelerometers to at least two vertebrae within the spine, and communicating the plurality of accelerometers with the vertebral processor.

The step of providing an external system includes aligning a reader coupled to a belt or strap over a position proximate to the implantable electronics assembly.

The step of collecting data of the relative movement of the vertebrae sensed by the implantable electronics assembly includes recording data received from the plurality of accelerometers coupled to the at least two vertebrae.

The step of calculating the relative orientation of the vertebrae further includes comparing the calculated relative orientation to a predetermined threshold value.

The method further includes the steps of determining the state of the relative movement of the spine by designating the status of the at least two vertebrae as a rigid body when the calculated relative orientation is consistent with the predetermined threshold value, or designating the status of the at least two vertebrae as an alarm condition when the calculated relative orientation received from the plurality of accelerometers is not consistent with the predetermined threshold value.

The method further includes the steps of communicating the determined state to the external system for review by transmitting the designated status of the at least two vertebrae to the external system through an inductive link between the external system and the implantable electronics assembly.

The method further includes the steps of calibrating the plurality of accelerometers after coupling them to the at least two vertebrae to create a patient specific data point.

The method further includes the steps of transcutaneously transmitting data and power between the external system and the implantable electronics assembly by an induction link.

The step of coupling the plurality of accelerometers coupled to the vertebral, processor to at least two vertebrae within the spine includes coupling at least one accelerometer above and at least one accelerometer below an interbody fusion cage or artificial disk.

The scope of the illustrated embodiments of the invention also includes a method for determining the success of a spinal fusion procedure which includes the steps of implanting a plurality of accelerometers and a vertebral processor with a plurality of implantable accelerometers coupled to at least two vertebrae in the spine of a patient, transmitting power to the vertebral body processor by an induction link from an external system proximate to the patient, sensing the relative acceleration of the plurality of accelerometers by an algorithm stored within the vertebral processor. If the relative acceleration of the plurality of accelerometers is equal to zero the at least two vertebrae are classified as a successful spinal fusion and if the relative acceleration of the plurality of accelerometers is beyond a predetermined threshold value, the at least two vertebrae are classified as an unsuccessful spinal fusion. The method also includes the step of communicating the fusion classification status to the external system.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

FIG. 1 is a diagram showing a lateral cross sectional view of the fusion sensing system in its relation to the spine of a patient.

FIG. 2a is a lateral lumbar view of the spinal column of a patient after the placement of spinal stabilization hardware and an interbody fusion cage, and before placement of the spinal sensing system.

FIG. 2b is a lateral lumbar view of the spinal column of a patient after placement of the spinal sensing system.

FIG. 3a is a posterior lumbar view of the spinal column of a patient after the placement of spinal fusion hardware and an interbody fusion cage, and before placement of the spinal sensing system.

FIG. 3b is a posterior lumbar view of the spinal column of a patient after placement of the spinal sensing system, the detector of the sensing system being mounted on the rod using mounting clamps.

FIG. 4a is a lateral lumbar view of the spinal column of a patient after placement of the spinal sensing system with the sensor located on the anterior spinal surface.

FIG. 4b is an anterior lumbar view of the spinal column of a patient after placement of the spinal sensing system with the sensor located on the anterior spinal surface, the detector of the sensing system being mounted on the L4 vertebra along with one of the sensors.

FIG. 5a is a lateral lumbar view of the spinal column of a patient after placement of the spinal sensing system without the presence of the pedicle screw and rod hardware with the sensors located on the posterior spinal region.

FIG. 5b is a lateral lumbar view of the spinal column of a patient after placement of the spinal sensing system without the presence of the pedicle screw and rod hardware with the sensors located on the anterior spinal region.

FIG. 5c is a lateral lumbar view of the spinal column of a patient after placement of the spinal sensing system on the anterior spinal region in conjunction with a placed artificial disc.

FIG. 6a is a lateral view of two successfully fused vertebrae and shows the positions of the upper accelerometer and lower accelerometer attached to adjacent vertebrae and used to detect the condition of the disc located between them.

FIG. 6b is a lateral view of two vertebrae with an unsuccessful fusion, the placed sensors, and their coordinate frames.

FIG. 7 is a schematic diagram of two cylinders representing the two vertebrae superimposed on each other to detect the difference in tilt angles. The coordinate frames for the two vertebrae are given by the gravity vector which is pointing in the −j direction.

FIG. 8 is a schematic diagram of the two cylinders representing the two vertebrae forming a joint. When the joint bends, a tilt angle can be calculated.

FIG. 9 is a flowchart of the operation of the sensor software and its three main stages.

FIG. 10 is a block diagram of the circuitry whereby power and data are transferred inductively from the external electronics unit in the belt to the implanted device.

FIG. 11 is a schematic circuit diagram of the inductive power circuits contained within the implanted device as well as in the external electronics unit in the belt.

FIG. 12 is an orthographic view of the external electronics unit mounted on a belt.

FIG. 13 is an orthographic view of the electronic circuits contained within the implant comprising the electronics connected to the inductive coils and a motion sensor.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

Unless specifically stated otherwise, all technical and scientific terms used herein have the same meanings as commonly understood by one of ordinary skill in the art to which this invention belongs. The following definitions are provided for illustration and clarity and not by way of limitation. The following terms retain their full scope of meaning as provided in the English language and/or as may be translated and the technical usage of the terms.

“Accelerometer” as used herein refers to a device that measures proper acceleration, the acceleration experienced relative to freefall. Single- and multi-axis models are available to detect magnitude and direction of the acceleration as a vector quantity, and can be used to sense position, vibration and shock.

“Acute Infection” as used herein refers to an infection with a rapid onset and/or a short course, or duration.

“Annulus Fibrosis” as used herein refers to the laminae of fibrous tissue and fibrocartilage each intervertebral fibrocartilage is composed of at its circumference.

“Anterior Lumbar Fusion” as used herein refers to an operation done on the front (the anterior region) of the lower spine. Fusion surgery helps two or more bones grow together into one solid bone. Fusion cages are new devices, essentially hollow screws filled with bone graft, that help the bones of the spine heal together firmly. Surgeons use this procedure when patients have symptoms from disc degeneration, disc herniation, or spinal instability.

“Anterior Lumbar Interbody Fusion Device” as used herein refers to the allograft spacers similar to those produced by Synthes North America to meet the specific demands of spinal applications.

“Artificial Disc” as used herein refers to an object used to replace an intervertebral disc when it is degenerated or when trying to fuse two vertebrae together. An interbody spinal cage is commonly used.

“Autograft” as used herein refers to the transplantation of organs, tissues or even proteins from one part of the body to another in the same individual. Tissue transplanted by such “autologous” procedure is referred to as an autotransplant.

“Bone Morphogenetic Proteins (BMP)” as used herein refers to a group of growth factors and cytokines known for their ability to induce the formation of bone and cartilage.

“Chronic infection” as used herein refers to an infection that exists in the host for a long period of time and generally has a slow onset.

“Comprising” as used herein refers to including, but not limited to, whatever follows the word “comprising”. Thus, use of the term “comprising” indicates that the listed elements are required or mandatory, but that other elements are optional and may or may not be present. Additionally, unless otherwise noted, exemplary lists of compounds or devices should not be construed as limiting; instead, it should be understood that such lists admit to additional, suitable items not explicitly indicated.

“Corpectomy” as used herein refers to a surgical procedure that involves removing part of the vertebral body, usually as a way to decompress the spinal cord and nerves.

“Decortication” as used herein refers to a medical procedure involving the surgical removal of the surface layer, membrane, or fibrous cover of an organ or bone.

“Fusion Mass” as used herein refers to the body of bone that connects two previously separate vertebra in the spine together.

“Gyroscope” as used herein refers to a device for measuring or maintaining orientation, based on the principles of conservation of angular momentum. A mechanical gyroscope is essentially a spinning wheel or disk whose axle is free to take any orientation. This orientation changes much less in response to a given external torque than it would without the large angular momentum associated with the gyroscope's high rate of spin. Since external torque is minimized by mounting the device in gimbals, its orientation remains nearly fixed, regardless of any motion of the platform on which it is mounted.

“Hypertrophy” as used herein refers to the increase in the volume of an organ or tissue due to the enlargement of its component cells. It should be distinguished from hyperplasia, in which the cells remain approximately the same size but increase in number.

“Inductive Power” as used herein refers to powering electronics using electromagnetic induction. A supplying induction coil sends energy through inductive coupling to a receiving inductive coil in an electrical device, which utilizes the energy. Because there is a small gap between the two coils, inductive charging is one kind of short-distance wireless energy transfer.

“Interbody Spinal Cages” as used herein refers to rigid (i.e. titanium, PEEK, or allograft) spacers, usually cylindrical, that are placed in the disc space. The cages are porous and allow the bone graft to grow from the vertebral body through the cage and into the next vertebral body.

“Intervertebral Disc Arthroplasty” as used herein refers to a surgical procedure in which degenerated intervertebral discs in the spinal column are replaced with artificial ones in the lumbar (lower) or cervical (upper) spine; also called Artificial Disc Replacement (ADR), or Total Disc Replacement (TDR).

“Magnetometer” as used herein refers to a scientific instrument used to measure the strength and/or direction of the magnetic field in the vicinity of the instrument. Magnetism varies from place to place and differences in Earth's magnetic field.

“Microelectromechanical systems (MEMS)” as used herein refers to the technology of the very small, and merges at the nano-scale into nanoelectromechanical systems (NEMS) and nanotechnology. MEMS are made up of components between 1 to 100 micrometers in size and MEMS devices generally range in size from 20 micrometers to a millimeter. They usually consist of a central unit that processes data, the microprocessor and several components that interact with the outside such as microsensors.

“Myelography” as used herein refers to a type of radiographic examination that uses a contrast medium to detect pathology of the spinal cord, including the location of a spinal cord injury, cysts, and tumors. The procedure often involves injection of contrast medium into the cervical or lumbar spine, followed by several X-ray projections. A myelogram may help to find the cause of pain not found by an MRI or CT.

“Osteoblasts” as used herein refers to mononucleate cells that are responsible for bone formation; in essence, osteoblasts are sophisticated fibroblasts that express all genes that fibroblasts express, with the addition of the genes for bone sialoprotein and osteocalcin. Osteoblasts produce osteoid, which is composed mainly of Type I collagen. Osteoblasts are also responsible for mineralization of the osteoid matrix. Zinc, copper and sodium are some of the many minerals produced. Bone is a dynamic tissue that is constantly being reshaped by osteoblasts, which build bone, and osteoclasts, which resorb bone.

“Osteoconduction” as used herein refers to the ability to stimulate the attachment, migration, and distribution of vascular and osteogenic cells within the carrier matrix material.

“Osteogenic” as used herein refers to the ability to generate or stimulate bone growth.

“Pars Articularis” as used herein refers to the part of the vertebra which lies behind the vertebral body and articulates with the adjacent vertebrae.

“Pedicle Screw” as used herein refers to a means of gripping a spinal segment. The screws themselves do not fixate the spinal segment, but act as firm anchor points that can then be connected with a rod. The screws are placed at two or three consecutive spine segments and then a short rod is used to connect the screws. This construct prevents motion at the segments that are being fused.

“PEEK” as used herein refers to Polyether-etherketone, a hard radiolucent plastic that is used in conjunction with carbon fiber reinforcement or as pure PEEK. Most manufacturers who use PEEK use radio marker dots so the surgeon can see where the implant meets the vertebral body endplate. Numerous companies (Zimmer Spine, Surgicraft, SCIENT'X, and Depuy Spine) have all developed lines of interbody fusion devices using PEEK technology.

“Posterior Lumbar Fusion” as used herein refers to an operation done on the back (the posterior region) of the lower spine. Fusion surgery helps two or more bones grow together into one solid bone. Fusion cages are new devices, essentially hollow screws filled with bone graft, that help the bones of the spine heal together firmly. Surgeons use this procedure when patients have symptoms from disc degeneration, disc herniation, or spinal instability.

“Pseudarthrosis” as used herein refers to the movement of a bone at the location of a fracture resulting from inadequate healing of the fracture. Pseudarthrosis can also result from a developmental failure.

“Subacute infection” as used herein refers to an infection that has an onset and course that is longer than that of an acute infection but also shorter than a chronic one.

### DETAILED DESCRIPTION OF THE PREFERRED EMBODIMENTS

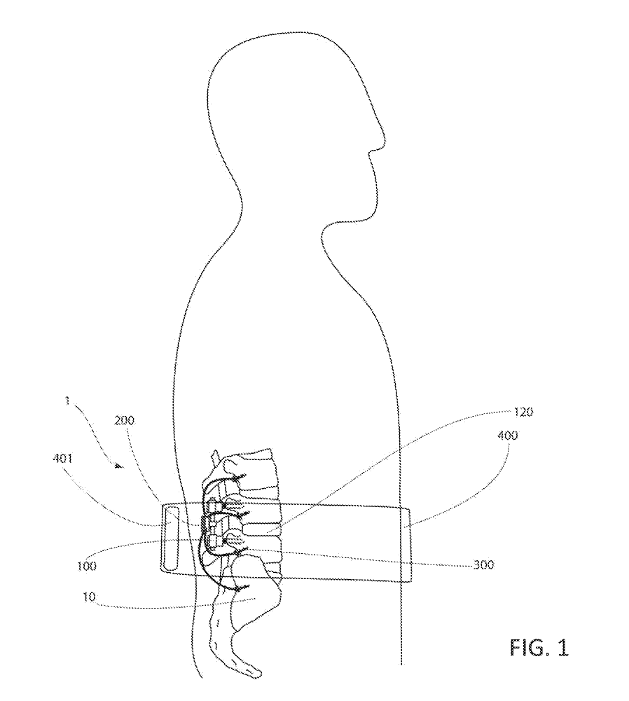

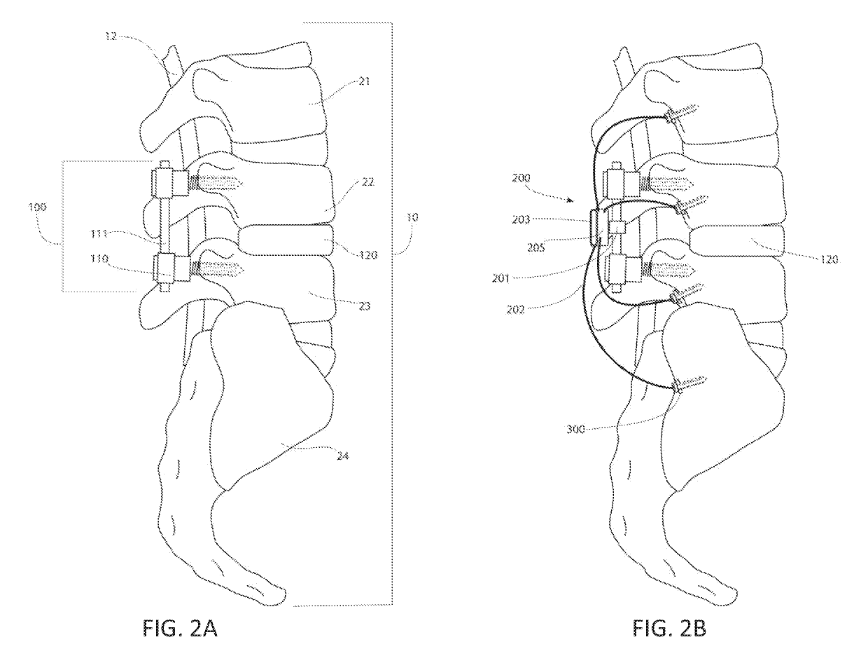

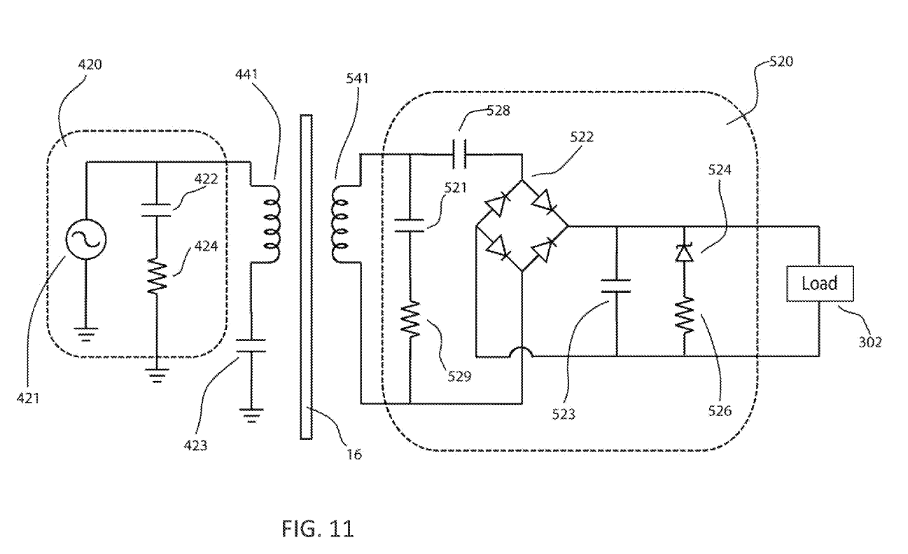

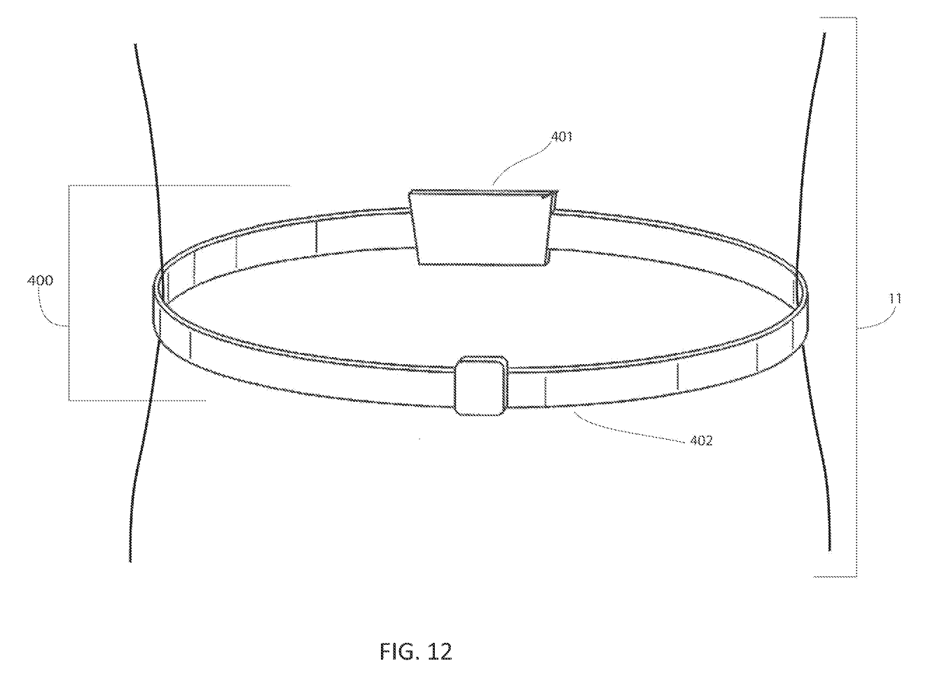

FIG. 1 is a lateral cross sectional view of a fusion sensing system 1 in its relation to the spine 10 of a patient. In this embodiment, the fusion sensing system 1 comprises an implant electronics assembly, generally denoted by reference numeral 200, coupled to spine stabilization hardware assembly, generally denoted by reference numeral 100, for interbody fusion of L4 and L5 discs of the lumbar spine using an interbody cage 120 and external wearable system 400. The fusion sensing system 1 couples a plurality of motion sensors 300 mounted into the spine 10 as best seen in FIG. 2b. The fusion sensing system 1 is powered via induction coils by a reader 401 coupled to the wearable system 400 that is worn externally by the patient. The reader 401 also comprises means for communicating to the implant electronics assembly 200 via the inductive coupling or link between the induction coils 441, 541 in FIGS. 11 and 12.

FIG. 2a is a lateral lumbar view of the spinal column 10 of a patient after the implantation of the spinal stabilization hardware 100 and the interbody fusion cage 120, but before implantation of the implant electronics assembly 200. The spinal stabilization hardware 100 comprises a plurality of pedicle screws 110 linked together by a stabilization rod 111. In this particular embodiment, the fusion cage 120 is in between L4 vertebra 22 and L5 vertebra 23. However the specific position of implantation of the fusion cage 120 and pedicle screws 110 shown in FIG. 2a are meant to be for illustrative purposes only. It would be clear to one skilled in the art that other positions within other vertebra may be used without departing from the original spirit and scope of the invention.

FIG. 2b is a lateral lumbar view of the spinal column of a patient after placement of the implant electronics assembly 200. The implant electronics assembly. 200 comprises a detector or vertebral body motion sensor (VBMS) 205 which is coupled to the rod 111 using at least one mounting clamp 201. The VBMS 205 itself comprises the internal electronics, a plurality of induction coils 203 for power and communication, and is coupled to a plurality of connecting wires 202 which are in turn coupled to the corresponding plurality of motion sensors 300 screwed into the vertebrae 21, 22, 23, 24 of the patient. Note that the plurality of induction coils 203 is likewise the embodiment of the induction coil for power receiving in implantable system 541 and induction coil for data transmission in implantable system 542.

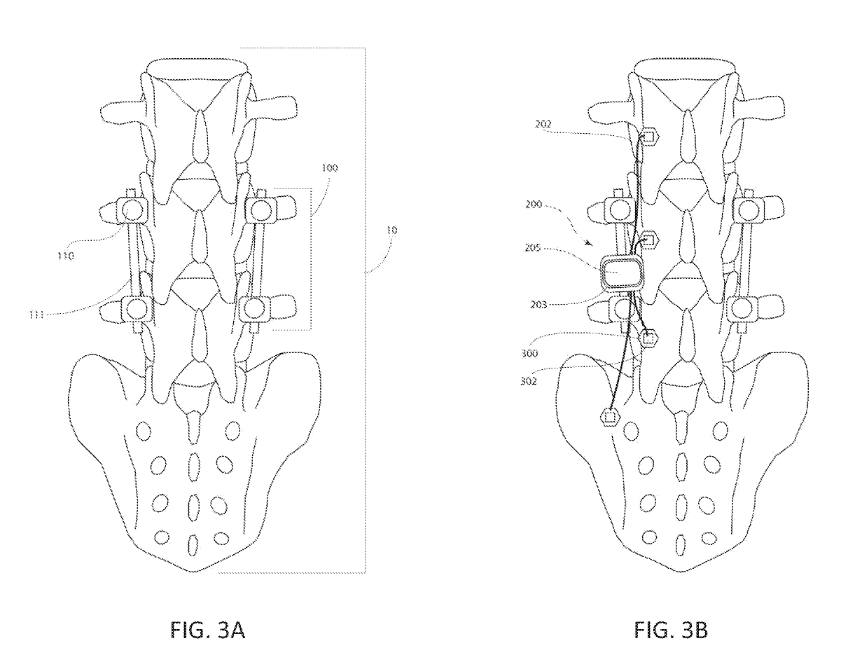

FIGS. 3a and 3b are posterior lumbar views of the embodiments illustrated in lateral lumbar view in FIGS. 2a and 2b. FIG. 3a is a posterior lumbar view of the spinal column 10 of a patient after the placement of spinal fusion hardware 100 and interbody fusion cage 120, but before placement of the implant electronics assembly 200. The spinal stabilization hardware 100 comprises of a plurality of pedicle screws 110 linked together by the stabilization rod 111. In this embodiment, the fusion cage is in between L4 vertebra 22 and L5 vertebra 23.

FIG. 3b is a posterior lumbar view of the spinal column 10 of a patient after placement of the implant electronics assembly 200. The VBMS 205 of the implant electronics assembly 200 is coupled to the rod 111 using mounting clamps 201. The VBMS 205 comprises the internal electronics, the induction coils 203 for power and communication, and is coupled to the plurality of connecting wires 202 which are in turn coupled to the corresponding plurality of motion sensors 300 screwed into the vertebrae 21, 22, 23, 24 of the patient.

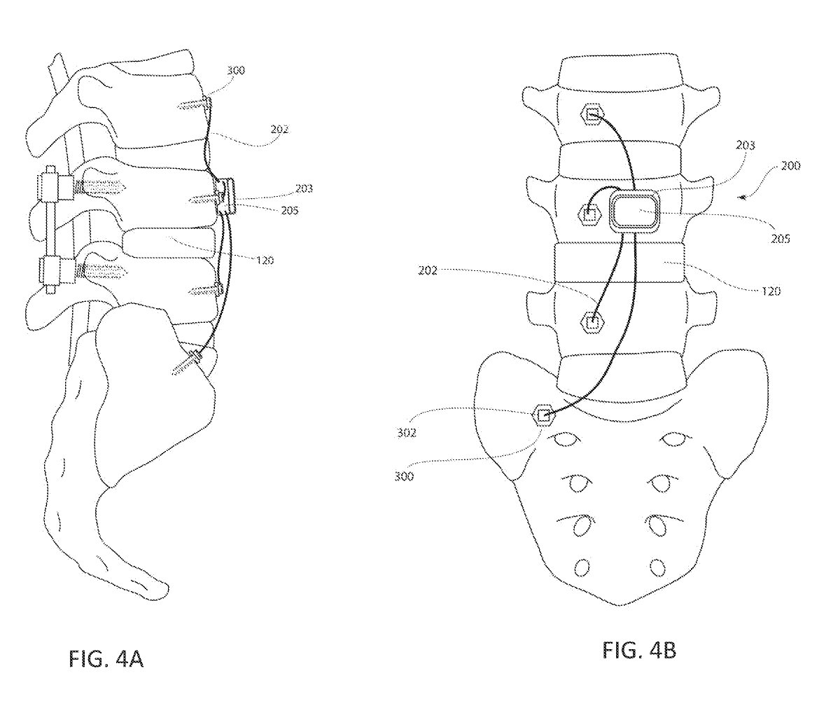

FIG. 4a is a lateral lumbar view of the spinal column 10 of a patient after placement of the implant electronics assembly 200 in an alternative embodiment with the sensors 300 coupled to the anterior spinal surface. The VBMS 205 is mounted on the L4 vertebra as is one of the plurality of sensors 300 within the implant electronics assembly 200. The VBMS 205 comprises the internal electronics, the induction coils 203 for power and communication, and is coupled to the plurality of connecting wires 202 which are in turn coupled to the corresponding plurality of motion sensors 300 screwed into the vertebrae 21, 22, 23, 24 of the patient.

FIG. 4b is an anterior lumbar view of the spinal column 10 of a patient after placement of the implant electronics assembly 200 in an alternative embodiment with the sensors 300 located on the anterior spinal surface. The VBMS 205 is mounted on the L4 vertebra as is one of the plurality of sensors 300 within the implant electronics assembly 200. The VBMS 205 comprises the internal electronics, the induction coils 203 for power and communication, and is coupled to the plurality of connecting wires 202 which are in turn coupled to the corresponding plurality of motion sensors 300 screwed into the vertebrae 21, 22, 23, 24 of the patient.

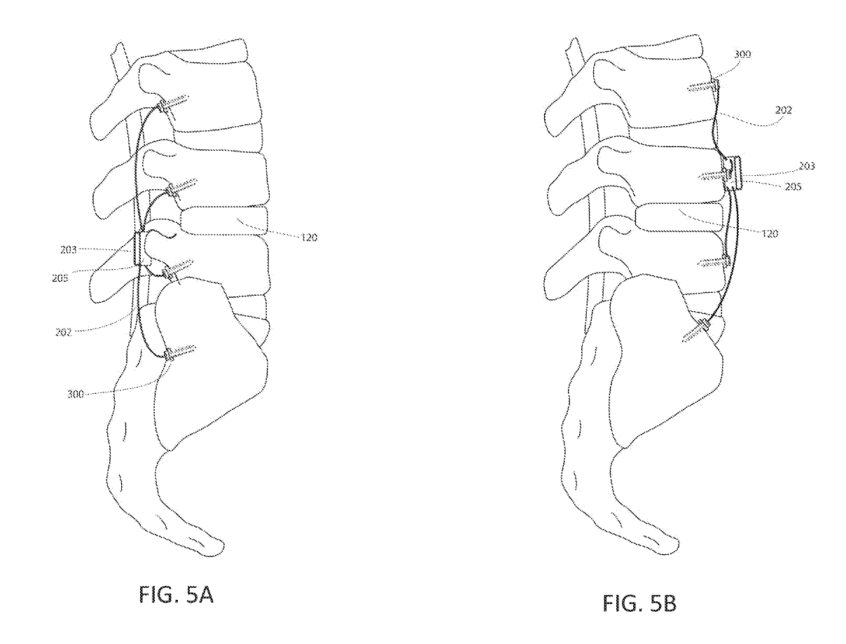

FIG. 5a is a lateral lumbar view of the spinal column 10 of a patient after placement of the implant electronics assembly 200 in another alternative embodiment without the presence of the pedicle screws 110 and rods 111 with the sensors 300 located on the posterior spinal region. The VMBS 205 is mounted on the L5 vertebra as is one of the plurality of sensors 300 within the spinal sensing system 1. The VBMS 205 comprises the internal electronics, the induction coils 203 for power and communication, and is coupled to the plurality of connecting wires 202 which are in turn coupled to the corresponding plurality of motion sensors 300 screwed into the vertebrae 21, 22, 23, 24 of the patient.

FIG. 5b is a lateral lumbar view of the spinal column 10 of a patient after placement of the implant electronics assembly 200 in an alternative embodiment on the anterior spinal region without the presence of the pedicle screws 110 and rods 111 with the sensors 300 located on the anterior spinal region. The VBMS 205 is mounted on the L5 vertebra as is one of the plurality of sensors 300 within the implant electronics assembly 200. The VMBS 205 comprises the internal electronics, the induction coils 203 for power and communication, and is coupled to the plurality of connecting wires 202 which are in turn coupled to the corresponding plurality of motion sensors 300 screwed into the vertebrae 21, 22, 23, 24 of the patient.

FIG. 5c is a lateral lumbar view of the spinal column 10 of a patient with an implanted artificial disc 35 after placement of the implant electronics assembly 200 on the anterior spinal region without the presence of the pedicle screws 110 and rods 111. The VBMS 205 is mounted on the L5 vertebra as is one of the plurality of sensors 300 within the implant electronics assembly 200. The VBMS 205 is mounted on the L5 vertebra as is one of the plurality of sensors 300 within the implant electronics assembly 200. The VBMS 205 comprises the internal electronics, the induction coils 203 for power and communication, and is coupled to the plurality of connecting wires 202 which are in turn coupled to the corresponding plurality of motion sensors 300 screwed into the vertebrae 21, 22, 23, 24 of the patient. In this configuration, the motion of the vertebrae on either side of the artificial disk 35 would be analyzed in the same way as vertebrae motion for adjacent disc disease as will be further detailed below.

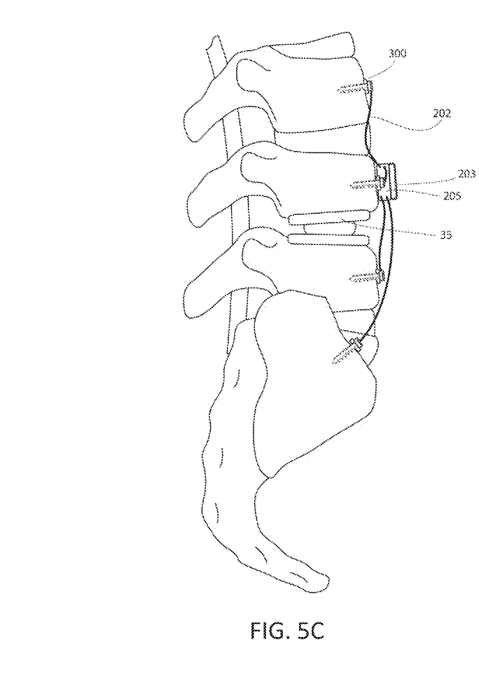

FIGS. 6a and 6b show the positions of two sensors 300, specifically in this embodiment, an upper accelerometer 312 and a lower accelerometer 313 coupled to adjacent vertebrae and used to detect the condition of the disc located between them. FIG. 6a is a lateral view of two successfully fused vertebrae 17, the placed sensors or accelerometers 312, 313, and their respective coordinate frames 310, 311. When the fusion is successful, the acceleration measurements from upper accelerometer 312 and lower accelerometer 313 should also conform to the dynamic physics of rigid body systems. The coordinate frames 310, 311 may be calibrated so that the difference in tilt angles between is negligible. The monitoring of relative movement between the upper accelerometer 312 and the lower accelerometer 313, i.e., vibrational movement, would also produce negligible differences in acceleration between the two sensors 312, 313 because the two successfully fused vertebrae 17 would form a single rigid body.

FIG. 6b is a lateral view of two vertebrae with an unsuccessful fusion 18. Here, the difference in tilt angles between the coordinate frame 310 of the upper accelerometer 312 and the coordinate frame 311 of the lower accelerometer 313 is detectable when the patient is bending. In addition, monitoring of relative movement between the upper accelerometer 312 and the lower accelerometer 313, i.e., vibrational movement, will detect the difference in acceleration because the two vertebrae will not be moving as one rigid body but rather as two separate elements.

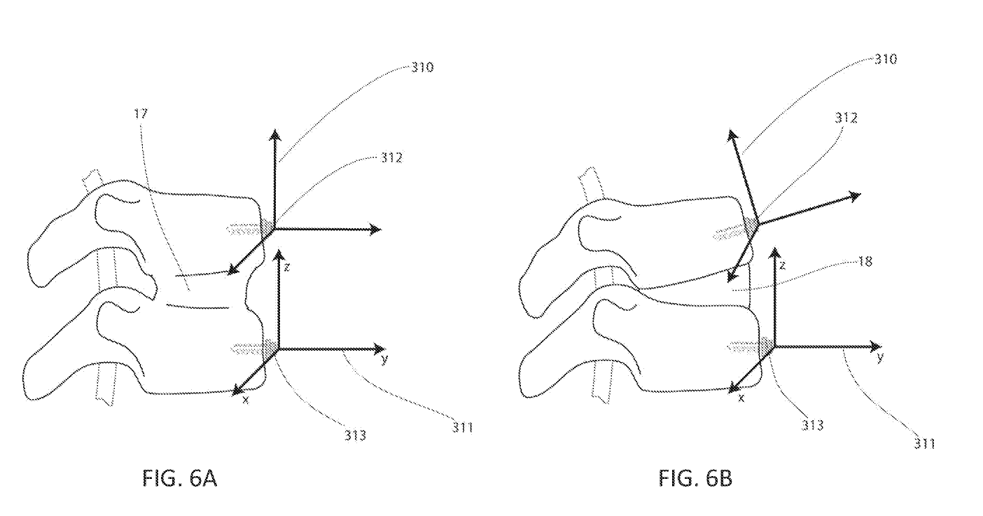

FIG. 7 illustrates how the accelerometer is used to measure its tilt relative to the gravity vector, g̅. The accelerometer represents g̅ in terms of its local orthogonal reference frame (î, ĵ, \hat{k}). This orthogonal representation is converted to the spherical coordinate system, to obtain the tilt angles (φ and θ), using the following formulas:

$$\{ \begin{matrix}cos φ = \overline{g} \cdot \hat{j} & \\ tan θ = \frac{{\overline{g}}_{x - z} \cdot \hat{i}}{{\overline{g}}_{x - z} \cdot \hat{k}} , & where {g}_{x - z} = ( \hat{j} \times \overline{g} ) \times \hat{j}\end{matrix}$$

g̅ is the gravity or acceleration vector in a local frame, e.g. 310. On a static rigid body such as that shown in FIG. 6a, the relative tilt angles (φ₁-φ₂ and θ₁-θ₂) between the two accelerometers 312, 313 are constant. This method is used to detect whether the fusion region 17 of FIG. 6a has deteriorated. It is also used to detect if two un-fused vertebrae are tilted to a dangerous degree (a sign of adjacent segment disease).

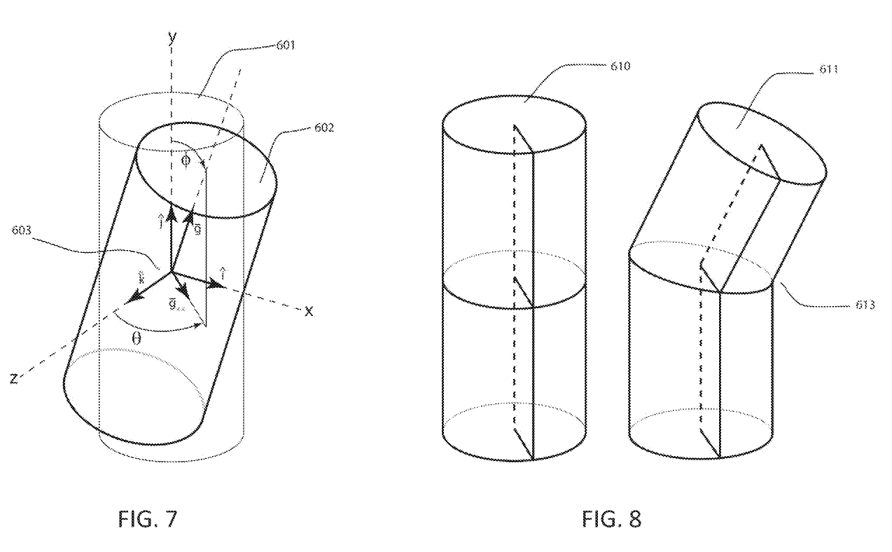

FIG. 8 is a schematic diagram of the two cylinders embodying the two vertebrae forming a joint 613. When the joint 613 bends, the upper accelerometer 312 in FIGS. 6a, 6b also bends with respect to the lower accelerometer 313 and thus a tilt angle can be calculated.

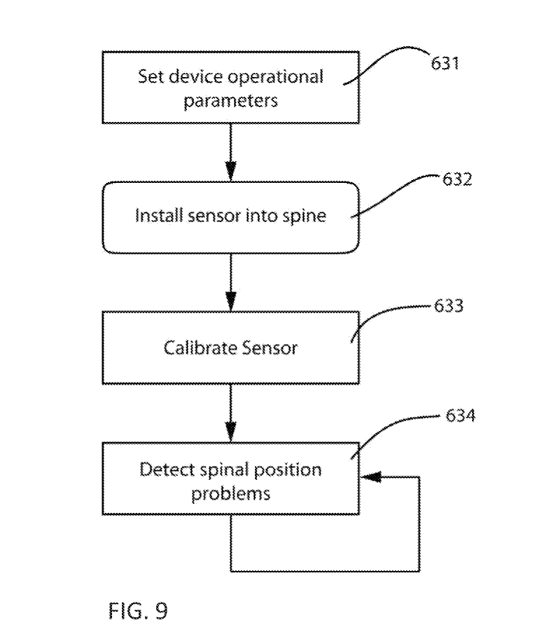

FIG. 9 is a flowchart of the operation of the spinal sensing system software which is comprised of three stages. In the first stage 631, the user programs the operational parameters of the spinal sensing system 1. This includes setting thresholds that specify what is considered to be error conditions. These parameters include, but are not limited to, sensor characteristics such as sensor measurement offset and noise, physical limits such as the maximum angular acceleration and maximum angular velocity, and boundary thresholds such as maximum φ and θ values to determine alarm conditions. This first stage is completed before implantation of the implant electronics assembly 200 in the patient.

Further shown in FIG. 9, the second stage 633 occurs after the implantation step 632 of the implant electronics assembly 200. The distance between the sensors or accelerometers 300 on the fused vertebrae 17 is programmed into the spinal sensing system 1 via the electronics contained within the VBMS 205. Next, the initial readings from each of the accelerometers 300 are taken and internally stored within a non-volatile memory contained within the VBMS 205. These values are kept as references to determine if future accelerometer readings fall within the acceptable range of motion of the spine 10.

Additionally as shown in FIG. 9, in the final stage 634, the spinal sensing system 1 periodically reads position information from each of the plurality of accelerometers 300 to determine if any problems are developing. To detect pseudarthrosis, the relative acceleration of at least two accelerometers 300 on the fused vertebrae 17 are used to determine if they are operating within the parameterized thresholds set for rotational motion, vibrational motion, and tilt angles. To detect adjacent segment disease, the readings from at least two sensors 300, for example the upper accelerometer 312 the lower accelerometer 313 coupled to the vertebrae above and below the fusion 17 are used to ensure that tilt angles do not exceed the specified threshold.

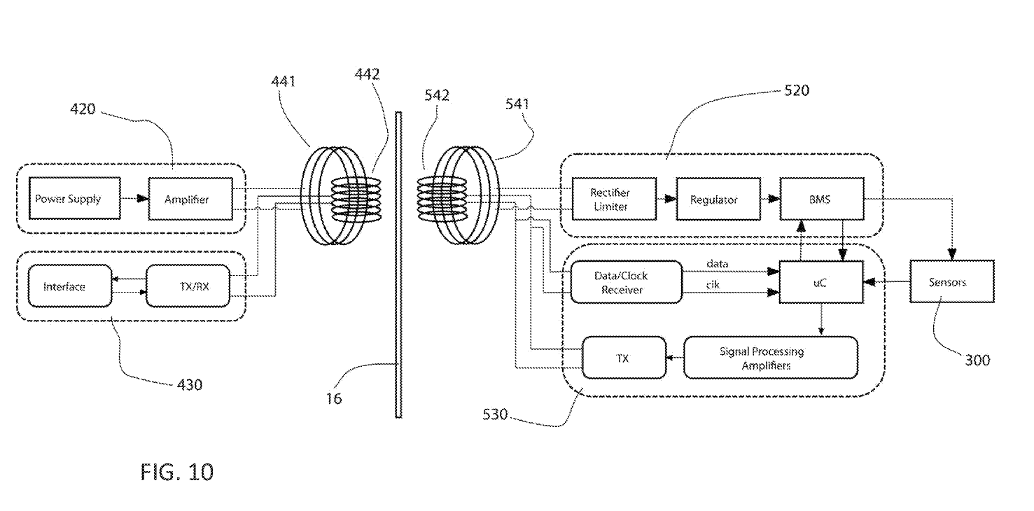

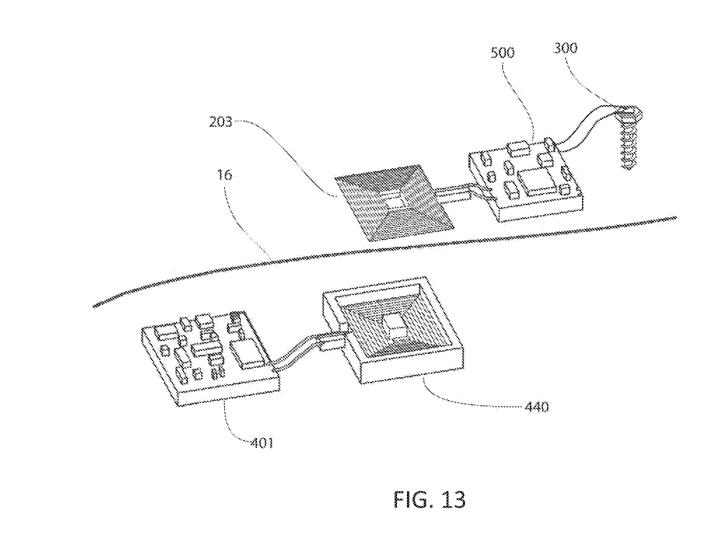

FIG. 10 is a block diagram of the circuit for inductively providing power and data transfer from the external wearable system 400 to the implant electronics assembly 200 of the spinal sensing system 1. A plurality of belt inductive coils 441 and a plurality of implant inductive coils 541 are used to transfer power transcutaneously through the skin 16 of the patient from an inductive power generation circuit 420 disposed within the external wearable system 400 to an implant power regulator circuit 520 disposed within the electronics of the VBMS 205 of the spinal sensing system 1. The implant power regulator circuit 520 provides power to a data communication circuit 530 and to the sensors 300. The power regulator circuit 520 and data communication circuit 530 together form an implant circuit 500 as seen in FIG. 13. A second set of inductive coils, namely belt data coils 442 and implant data coils 542 are used to transfer data transcutaneously through the skin 16 from the implant data circuit 530 to a data receiver circuit 430 disposed within the external wearable system 400. The data receiver circuit 430 and the power generation circuit 420 together form a belt circuit 401 as seen in FIG. 13.

FIG. 11 is a schematic circuit diagram of the electronic circuits comprising the power regulator circuit 520 and inductive power generation circuit 420. The inductive power generation circuit 420 contains an alternating current source 421 biased by a capacitor 422 and a resistor 424. The power from the circuit is radiated through a matched resonance circuit comprised of the belt induction coils 441 and a capacitor 423. The power transmitted through the skin 16 is received by the implant induction coils 541 which energizes the implant power regulatory circuit 520 which comprises a capacitor 521 and a resistor 529 to match the resonance. The voltage is high pass filtered by a capacitor 528 before being rectified by a diode bridge 522. The rectified signal is further low pass filtered by a capacitor 523 and shunt regulated by a Zener diode 524 and a resistor 526 before powering a load circuit 302 electrically coupled to the plurality of sensors.

FIG. 12 is an orthographic view of the external wearable system 400 comprising a reader unit 405 mounted on a belt 402. The belt 402 which houses the reader 405 is worn around the torso 11 of the patient so that the belt inductive coils 441 are as closely aligned with the implanted inductive coils 541 as possible.

FIG. 13 is an orthographic view of the electronic circuits contained within the implant electronics assembly 200 comprising the implant circuit 500 coupled to the plurality of inductive coils in the implantable system 203 and at least one sensor or accelerometer 300. The implant circuit 500 is charged through the skin 16 by the belt circuit 401 and plurality of inductive coils on the belt 440. An example of the makeup of the plurality of inductive coils on the belt can be found in FIG. 10, and is shown by the depiction of the combination of the induction coil for power transfer in external belt 441 and the induction coil for data receiving in external belt 442.

Relative Acceleration Between Two Points on a Rigid Body

Acceleration between two different points (Points 1 and 2) has the relationship as shown in Equation 1.

ā_{2/1}=ā₂−ā₁ (1)

Where ā_{2/1} is relative acceleration between the two points on a rigid body. For linear accelerations, ā_{2/1}= 0̅ since the acceleration experienced at both point 1 and point 2 are equal. For rotational motions, the two points will experience different accelerations. Their relative acceleration is independent of the center of rotation and given in Equation 2.

ā_{2/1}=ā_{tangental}+ā_{centripal}=( α̅× r̅_{2/1})−ω² r̅_{2/1} (2)

Where α̅ is angular acceleration given in radians/sec², ω is angular velocity given in radians/sec and r̅_{2/1} is the distance between the two points. By setting allowed maximum values for α̅ and ω, the operational bounds of the system 1 can be set. If the relative acceleration is measured to be outside this bound, we can assume that the rigid body assumption no longer holds.

Accelerometer Sensor Output

A triaxial accelerometer measures linear acceleration in the orthogonal axes of the three dimensional space. The output vector of the sensor 300 is comprised of acceleration, gravity, an offset error, and measurement noise component vectors, as shown in Equation 3.

s̅=ā+ g̅+ē+ n̅ (3)

Where s is the sensor output vector in three dimensional space, ā is the acceleration component, g̅ is the gravity component, ē is the sensor offset error due to manufacturing technique, and n̅ is white noise due to the measuring environment and process. All of these vectors are expressed in the sensor coordinate frame 310, 311. Although the offset error and white noise can slowly drift over time, it is usually assumed constant and the sensors 300 are calibrated periodically to update these constants. The gravity component vector indicates the inclination of the sensor coordinate frame 310, 311 with respect to the earth's gravity field. In turn, the presence of the gravity vector helps the sensor calibration when the sensor 300 is stationary.

In order to detect acceleration experienced by the sensor 300, the estimated gravity, offset, and noise components are subtracted from the sensor output, as shown in Equation 4.

ā= s̅−( g̅+ē+ n̅) (4)

Vibration Monitoring

For a non-rotating, vibrating rigid body, the angular rotation will be negligible compared to linear acceleration. The relative linear acceleration between two sensors on a rigid body can be expressed as:

ā_{2/1}=ā₂−ā₁=( s̅₂− s̅₁)+( g̅₁− g̅₂)+(ē₁−ē₂)+( n̅₁− n̅₂) (5)

By setting the coordinate frames of the two sensors the same, the two gravity terms g̅₁ and g̅₂ can be calibrated to be equal. The two offset terms (ē₁ and ē₂) can also be calibrated to zero, reducing Equation 5 to Equation 6.

ā_{2/1}=( s̅₂− s̅₁)=( n̅₂− n̅₁) (6)

The noise can be obtained and the system can be calibrated to ignore the readings that follow the inequality described in Equation 7.

| s̅₂− s̅₁|<| n̅₂|+| n̅₁| (7)

If the body becomes non-rigid, the vibration response of the two sensors will be significantly larger. In addition, the two gravity vectors are not necessarily in the same direction, leading to a non-zero difference between the gravity terms. Thus, the relative acceleration between two points on a non-rigid body becomes significantly larger, as shown in Equation 7.

|ā_{2/1}|=|( s̅₂− s̅₁)+( g̅₁− g̅₂)|>>| n̅₂|+| n̅₁| (8)

Tilt Angle Monitoring

When stationary, a triaxial accelerometer can easily determine the sensor's orientation respect to the gravity by detecting the gravity vector in its coordinate frame. The gravity vector can be represented in the spherical coordinate system (| g̅|, φ, θ) by transforming the three component axes (î, ĵ, \hat{k}) as shown in Equations 9-11.

$$\begin{matrix}\vert \overline{g} \vert = \sqrt{{( \overline{g} \cdot \hat{i} )}^{2} + {( \overline{g} \cdot \hat{j} )}^{2} + {( \overline{g} \cdot \hat{k} )}^{2}} & ( 9 ) \\ \vert \overline{g} \vert cos φ = \overline{g} \cdot \hat{j} & ( 10 ) \\ tan θ = \frac{{\overline{g}}_{x - z} \cdot \hat{i}}{{\overline{g}}_{x - z} \cdot \hat{k}} & ( 11 )\end{matrix}$$

where g̅_{x-z} is the projection of gravity vector onto the x-z plane given by g̅_{x-z}=(ĵ× g̅)×ĵ.

By using gravity as the common reference between the two sensors, for example, the upper accelerometer 312 and the lower accelerometer 313, the difference between tilt angles (φ and θ) are constant for two points on a rigid body.

Surgical Implantation for Spinal Fusions

The spinal sensing system 1 is made for the purpose of determining the success of spinal fusion surgeries (anterior/posterior) and for artificial disc implants. For anterior fusions, it may be placed after an interbody graft 120 (bone/titanium cage) has been placed, with or without a plate as seen in FIGS. 4b and 5b. A drill is used to make a predetermined hole in the vertebral body above and below the interbody graft 120. The vertebral body motion sensor (VBMS) 205 is then screwed into the vertebral body as seen in FIG. 4a. The accelerometer signal wires (ASW) 202 are preconnected to the plurality of sensors 300 before implantation which are screwed into a corresponding plurality of vertebrae within the spine 10. In the case of a L4-5 anterior lumbar interbody fusion (ALIF) of FIGS. 4a-5c, the sensors 300 are screwed into at least the L4 and L5 vertebral bodies. The VMBS 205 is coupled either into L4 or L5 vertebral body as seen in FIG. 4b, and the signal wires 202 couple the at least two sensors 300 to the VBMS 205.

For a posterior fusion seen in FIGS. 2a-3b, after placement of the pedicle screws 110 and stabilization rods 111, the sensors 300 are placed into the pars articularis on either side of the fusion on each of the fused segments. The sensors 300 and the preconnected signal wires 202 are then coupled to the VBMS 205 which is clamped onto the stabilization rod 111. For a L4-5 posterior fusion, this procedure would entail placement of at least two sensors 300 into the pars articularis of the L4 and L5 vertebrae. The signal wires 202 would then be coupled to the VBMS 205 which is clamped onto the stabilization rod 111 between L4 and L5.

Surgical Implantation for Adjacent Segment Motion

For detection of adjacent segment motion for posterior fusions seen in FIGS. 2a-3b, the plurality of sensors 300 are implanted into the vertebrae above 22 and below 23 the fusion site at the level of the pars articularis.

For detection of adjacent segment motion for anterior fusions seen in FIGS. 4a-5c, the plurality of sensors 300 are implanted for L5-S1 ALIFs, with placement of the sensors 300 into the L4 vertebral body. For L4-5 ALIFs, the sensors 300 will have to be implanted into the L3 and S1 vertebral body. These levels are definitely harder to reach via an anterior exposure; however, it should be possible with retraction of the aorta, inferior vena cava, or internal iliac veins at the time of placement.

For detection of adjacent segment motion after artificial disc 35 placement seen in FIG. 5c, the plurality of sensors 300 will be implanted as described for anterior fusions. The cervical spine should have no problem with placement of the sensors 300 in the segments above and below the fusion site. The lumbar spine may have a problem with a L4-5 artificial disc (access may be limited for L3), but should be accessible for a L5-S1 artificial disc. Patients with artificial discs 35 may also have a sensor 300 implanted posteriorly at the levels of the pars articularis above and below the artificial disc 35. This procedure, however, requires another incision.

Postoperative Care

After the surgery is performed, the patient will wear an external brace as is normal practice. The reader 405 coupled to the belt 402 is placed much like a bone stimulator over the brace. The reader 405 comprises means for detecting motion over the fusion segment and detecting flexion/extension motion in the adjacent segments as discussed above. The reader 405 houses a power source with inductive coils as seen in FIG. 10, and contains the wireless accessible memory chip that will be fed back to the surgeon for detection of spinal motion.

Use of the Accelerometer for Detecting Pseudarthrosis and Adjacent Segment Disease

If a patient presents with continued back pain after a spinal fusion, the reader 405 is placed over the implant electronics assembly 200. Ideally, the reader 405 will be present immediately after surgery to obtain a baseline reading. Over time, if there is increased motion detected over the fused segments, the diagnosis of pseudarthrosis may be made, if there is radiographic correlation.

For adjacent segment disease, increased motion on flexion/extension may be detected at the levels above and below the fused segments or artificial discs 35. The following example represents one embodiment of the invention. The example presented below in pseudocode compares adjacent accelerometer sensor 300 data. However, nonadjacent implanted accelerometer 300 data, for example comparing an implanted accelerometer sensor on L3 with an implanted accelerometer sensor on L5, may also be compared to provide relative position feedback without departing from the original spirit and scope of the invention. This example is meant for illustrative purposes only, and should not be construed to encapsulate the complete embodiment of this invention.

Global variables

| /* Parameters to be set before implantation */ | |
|---|---|
| Phi_upper_thresh | maximum relative flexion angle allowed |
| Om_upper_thresh | maximum relative lateral bending allowed |
| Phi_lower_thresh | maximum relative flexion angle allowed |
| Om_lower_thresh | maximum relative lateral bending allowed |
| Phi12_thresh | maximum relative flexion angle allowed |
| Om12_thresh | maximum relative lateral bending allowed |
| MaxAngAccel | maximum allowed angular acceleration |
| MaxAngVel | maximum allowed angular velocity |
| Noise1 | noise of sensor 1 (on fused vertebrae) |
| Noise2 | noise of sensor 2 (on fused vertebrae) |
| Offset1 | calibration offset of sensor 1 (on fused vertebrae) |
| Offset2 | calibration offset of sensor 2 (on fused vertebrae) |
| /* Parameters set during device calibration */ | |
| Phi_upper | relative flexion angle between sensor 1 and sensor on vertebrae |
| | above fused vertebrae |
| Om_upper | relative lateral bending between sensor 1 and sensor on |
| | vertebrae above fused vertebrae |
| Phi_lower | relative flexion angle between sensor 2 and sensor on vertebrae |
| | below fused vertebrae |
| Om_lower | relative lateral bending between sensor 2 and sensor on |
| | vertebrae below fused vertebrae |
| Phi12 | relative flexion angle between sensors on fused vertebrae |
| Om12 | relative lateral bending between sensors on fused vertebrae |
| r12 | distance between sensors on fused vertebrae |

Function:
(phi, om) = CalculateTilt(x, y, z)
Algorithm:
/* this function calculates the tilt of the system */
/* given the 3D accelerometer readings (x,y,z) */
mag = sqrt ( x\hat{ }2 + y\hat{ }2 + z\hat{ }2 );
phi = acos ( y / mag );
om = atan ( −x / z );
Return ( phi, om )
Function:
CalibrateTilt( (s_1_x, s_1_y, x_1_z),

(s_2_x, s_2_y, x_2_z),
(s_up_x, s_up_y, x_up_z),
(s_low_x, s_low_y, x_low_z))

Algorithm:
/* this function calculates the initial tilt of the system */
/* immediately after device implantation and used as reference */
(phi1, om1) = CalculateTilt ( s_1_x, s_1_y, s_1_z )
(phi2, om2) = CalculateTilt ( s_2_x, s_2_y, s_2_z )
(phi_up, om_up) = CalculateTilt ( s_up_x, s_up_y, x_up_z )
(phi_low, om_low) = CalculateTilt ( s_low_x, s_low_y, x_low_z )
Phi12 = phi2 − phi1
Om12 = om2 − om1
Phi_upper = phi1 − phi_up
Om_upper = om1 − om_up
Phi_lower = phi_low − phi2
Om_lower = om_low − om2

Return

Function:
CheckPseudoarthorsis( (s_1_x, s_1_y, x_1_z),

(s_2_x, s_2_y, x_2_z))

Algorithm:
ax = s_2_x − s_1_x
ay = s_2_y − s_1_y
az = s_2_z − s_1_z
maga = sqrt ( ax\hat{ }2 + ay\hat{ }2 + az\hat{ }2 );
/* check for errors assuming rotational motion */
If (maga > MaxAngAccel * r12) Return ROT_ERR
If (maga > MaxAngVel\hat{ }2 * r12) Return ROT_ERR
/* check for errors assuming vibrations */
If (maga > noise1 + offset1 + noise2 + offset2) return VIB_ERR
/* check for change in tilt angles */
/* check only if there are no other forces acting on spine */
If (maga = 9.8 + epsilon) {
(phi1, om1) = CalculateTilt ( s_1_x, s_1_y, s_1_z )
(phi2, om2) = CalculateTilt ( s_2_x, s_2_y, s_2_z )
(phi_up, om_up) = CalculateTilt ( s_up_x, s_up_y, x_up_z )
(phi_low, om_low) = CalculateTilt ( s_low_x, s_low_y, x_low_z )
If (phi2 −phi1 −Phi12 > Phi12_thresh) Return TILT_ERR
If (om2 −om1 −Om12 > Om12_thresh) Return TILT_ERR
If (phi1 −phi_up−Phi_upper > Phi_upper_thresh)Return ASD_ERR
If (om1 −om_up −Om_upper > Om_upper_thresh) Return ASD_ERR
If (phi_low−phi2 −Phi_lower > Phi_lower_thresh)Return ASD_ERR
If (om_low −om2 −Om_lower > Om_lower_thresh) Return ASD_ERR
}
Return

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following invention and its various embodiments.

Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the invention is explicitly contemplated as within the scope of the invention.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptually equivalent, what can be obviously substituted and also what essentially incorporates the essential idea of the invention.

We claim:

1. An apparatus for sensing relative vertebral movement within a spine of a patient comprising:
an implantable electronics assembly coupled to at least one of a plurality of vertebrae within the spine for monitoring spatial orientation of at least one of the plurality of vertebrae; and
an external system disposable proximate to the implantable electronics assembly and comprising an induction link and a circuit communicated to the implantable electronics assembly through the induction link for communicating data related to the spatial orientation of at least one of the plurality of vertebrae.

2. The apparatus of claim 1 where the implantable electronics assembly comprises:
a vertebral processor; and
at least two sensors or accelerometers coupled to the vertebral processor and to at least two corresponding vertebrae within the spine.

3. The apparatus of claim 2 where the vertebral processor is coupled to a vertebra within the spine of the patient.

4. The apparatus of claim 2 in combination with a spinal stabilization rod where the vertebral processor is coupled to the stabilization rod coupled to at least two adjacent vertebrae within the spine of the patient.

5. The apparatus of claim 1 where the implantable electronics assembly comprises:
an implantable data circuit and
an implantable induction coil coupled to the implantable data circuit,
where the external system comprises:
a data receiver circuit;
an external induction coil coupled to the data receiver circuit;
where the implantable data circuit transmits data received by the implantable electronics assembly to the data receiver circuit through electromagnetic coupling of the external and implantable induction coils; and where the data receiver circuit transmits instructions to the implantable data circuit and implantable electronics assembly through electromagnetic coupling of the external and implantable induction coils.

6. The apparatus of claim 1 where the implantable electronics assembly comprises:
a power regulator circuit; and
at least one power induction coil coupled to the power regulator;
where the external system comprises:
a power generation circuit; and
at least one power induction coil coupled to the power generation circuit;
where the power generation circuit transmits power through the power induction coil to the power regulator circuit; and where the power regulator circuit receives power from the power generator circuit through the power induction coil to power the implantable electronics assembly.

7. The apparatus of claim 1 where the external system comprises a strap, a reader unit coupled to the strap which is worn by a patient so that the reader unit is proximate to the implantable electronics assembly implanted within the patient.

8. The apparatus of claim 7 where the reader unit comprises a power generation circuit and a data receiver circuit coupled to the external induction coil.

9. The apparatus of claim 2 in combination with an interbody fusion cage or artificial disk where the at least two sensors or accelerometers are coupled to at least one vertebra above and at least one vertebra below the interbody fusion cage or artificial disk.

10. A method for monitoring relative movement of vertebrae in a spine of a patient comprising:
providing an implantable electronics assembly for coupling to a plurality of vertebrae within the spine capable of sensing movement of the plurality of vertebrae relative to one another;
providing an external system for proximate monitoring of the patient;
collecting data of the relative movement of the vertebrae sensed by the implantable electronics assembly; and
calculating the relative orientation of the vertebrae from the collected data.

11. The method of claim 10 further comprising:
coupling a vertebral processor directly or indirectly to a vertebra within the spine;
coupling a plurality of accelerometers to at least two vertebrae within the spine; and
communicating the plurality of accelerometers with the vertebral processor.

12. The method of claim 10 where providing an external system comprises aligning a reader coupled to a belt or strap over a position proximate to the implantable electronics assembly.

13. The method of claim 11 where collecting data of the relative movement of the vertebrae sensed by the implantable electronics assembly comprises recording data received from the plurality of accelerometers coupled to the at least two vertebrae.

14. The method of claim 13 where calculating the relative orientation of the vertebrae further comprises comparing the calculated relative orientation to a predetermined threshold value.

15. The method of claim 14 further comprising determining the state of the relative movement of the spine by designating the status of the at least two vertebrae as a rigid body when the calculated relative orientation is consistent with the predetermined threshold value; or designating the status of the at least two vertebrae as an alarm condition when the calculated relative orientation received from the plurality of accelerometers is not consistent with the predetermined threshold value.

16. The method of claim 15 further comprising communicating the determined state to the external system for review by transmitting the designated status of the at least two vertebrae to the external system through an inductive link between the external system and the implantable electronics assembly.

17. The method of claim 11 further comprising calibrating the plurality of accelerometers after coupling them to the at least two vertebrae to create a patient specific data point.

18. The method of claim 10 further comprising transcutaneously transmitting data and power between the external system and the implantable electronics assembly by an induction link.

19. The method of claim 11 where coupling the plurality of accelerometers coupled to the vertebral processor to at least two vertebrae within the spine comprises coupling at least one accelerometer above and at least one accelerometer below an interbody fusion cage or artificial disk.

20. A method for determining the success of a spinal fusion procedure comprising:
implanting a plurality of accelerometers and a vertebral processor with a plurality of implantable accelerometers coupled to at least two vertebrae in the spine of a patient;
transmitting power to the vertebral body processor by an induction link from an external system proximate to the patient;
sensing the relative acceleration of the plurality of accelerometers by an algorithm stored within the vertebral processor,
where if the relative acceleration of the plurality of accelerometers is equal to zero the at least two vertebrae are classified as a successful spinal fusion and if the relative acceleration of the plurality of accelerometers is beyond a predetermined threshold value, the at least two vertebrae are classified as an unsuccessful spinal fusion; and
communicating the fusion classification status to the external system.
